(19) United States
(12) Patent Application Publication
(10) Pub. No.: US 2015/0110907 A1
(43) Pub. Date: Apr. 23, 2015

(54) METHOD OF TESTING THE ACTIVITY OF A POTENTIALLY ACTIVE SUBSTANCE TO INHIBIT THE ENZYMATIC ACTIVITY OF PHOSPHOLIPASE A2

(71) Applicant: BASF Beauty Care Solutions France S.A.S., Lyon (FR)

(72) Inventors: Eric Perrier, Les Cotes d'Arey (FR); Sébastien Bonnet, Saint Jean De Niost (FR); Delphine Rival, Ternay (FR)

(21) Appl. No.: 14/585,256

(22) Filed: Dec. 30, 2014

Related U.S. Application Data
(62) Division of application No. 10/365,897, filed on Feb. 12, 2003, now abandoned.

(30) Foreign Application Priority Data
Nov. 19, 2002 (FR) 02 14490

(51) Int. Cl. A61K 8/97; A61Q 19/00; A61K 36/87; A61K 8/37; A61K 36/488; A61K 8/34

(52) U.S. Cl. CPC A61K 8/97; A61K 36/488; A61K 8/345; A61K 36/87; A61K 8/37; A61Q 19/00; A61K 2800/782
USPC 424/757; 424/766

(57) ABSTRACT

The invention relates to a method of testing the activity of a potentially active substance to inhibit the enzymatic activity of phospholipase A2. The invention relates notably to a method wherein it comprises a) placing the potentially active substance in the presence with the phospholipase A2; a substrate which is a phospholipid, comprising at least one fatty acid in the form of an ester, the fatty acid is preferably a long chain having between 15 and 22 carbon atoms, this fatty acid preferably being unsaturated or poly-unsaturated, the substrate being capable of releasing at least one fatty acid during its hydrolysis; b) measuring the enzymatic activity of the phospholipase A2, notably comprising detecting the presence of the fatty acid and eventually its quantitative determination. The use of this test method notably enables identifying and selecting an active principle capable of inhibiting the enzymatic activity of phospholipase A2.

### RELATED APPLICATIONS

This application is a divisional of patent application Ser. No. 10/365,897 filed Feb. 12, 2003, which claims benefit of French application 02 14490 filed Nov. 19, 2002. The entire content of each aforementioned application is hereby incorporated by reference in its entirety.

The invention relates essentially to an active principle capable of reducing skin inflammation and to its use mainly in the field of cosmetics or pharmacy.

The present invention relates essentially to a method of testing a substance which is potentially active in the field of inflammation.

The present invention relates essentially to a novel test method and to its use, for the research and the identification of a substance which is potentially active in the field of inflammation, which is based on the capacity of inhibition of the enzyme phospholipase A2 (PLA2).

The present invention relates essentially to the novel substances which are active in the field of inflammation thus detected and to their use in the cosmetic or dermo-pharmaceutical or pharmaceutical field, notably for carrying out cares which enable reducing the signs of skin irritation.

### STATE OF THE ART

The anti-inflammatory products developed in laboratories to this day, which are intended to fight against skin inflammation, are selected on study models which are not adapted to the conditions which enable demonstrating the activities linked to the inflammation of the skin (rashes, scurf, xerosis, blotchiness . . . ).

The models used are in fact for the most developed on animals where the stress generated remains very drastic, since it can be:
a UV irradiation in a specific point, with the aim of generating a rash and of testing, after application of the potentially active product, the disappearance of this rash;
a subcutaneous injection of lipopolysaccharides (LPS) and/or guanidine and/or even of carrageen so as to create an edema in the guinea pig, and then to test, after application of the potentially active product, the disappearance of this edema.

In vitro tests are also developed which treat the inhibition of phospholipase A2, an enzyme involved in the synthesis of the mediators of inflammation, but these tests are very distant from the conditions encountered in vivo and are consequently not very significant.

Predose Details on the known In Vitro Tests:

The tests described up to now pose problems of sensitivity and are very distant from the situation encountered in vivo by the nature of the constituents of the models used. Thus, for example, the patent FR 2,757,395 A1 describes the inhibition of a phospholipase A2 allowed to react with a substrate which is very distant from the natural substrates of this enzyme which are encountered in inflammation reactions; this substrate, dimyristoyl L-phosphatidylcholine is in fact a phospholipid containing two C14 fatty acids (saturated hydrocarbon fatty chain of 14 carbon atoms). Now, this substrate and these fatty acids are not involved in the chain of formation of the mediators of inflammation since it is a phospholipid which carries an unsaturated C20 fatty acid (arachidonic acid) which is hydrolyzed by phospholipase A2 on this occasion.

Still within this document, the fatty acid in SN2 position hydrolyzed by the enzyme and the release of the fatty acid leads to the appearance of a cloudiness of the solution due to the insolubility of the fatty acid in the medium measured in spectrophotometry at 360 nm. This technique is not very sensitive and does not enable on the one hand obtaining a reliable classification of the inhibitors; on the other hand, the technique does not enable testing lipophilic or emulsifying molecules which, in making the solution cloudy, do not enable a correct measurement of the inhibition of the phospholipase A2.

### The A2 Phospholipases (PLA2s),

Phospholipase A2 is an enzyme which is produced by cells at the membrane level. It predominates in the cells which are linked to inflammation phenomena, such as the mastocytes. This enzyme hydrolyses the membrane phospholipids in type 2 nucleophilic substitution (SN2) position in order to release a fatty acid.

Schematically, 3 types of functions can be attributed to the phospholipases digestive functions, functions of restructuring membrane phospholipids, which are responsible for maintaining cell architecture, incorporating deacylation/reacylation cycles, and finally functions of transduction of signals by the production of biologically active products from membrane phospholipids thus, the regulation of the activation of the phospholipases will become a predominant element of the signal cascades and therefore of amplification of the signals during inflammatory reactions.

PLA2s have first of all been identified in the extracellular medium of various species mammals (pancreatic PLA2), snakes, insects (venom PLA2). Later on, five groups of PLA2s were defined, characterised both enzymatically and structurally and functionally (Table 1).

TABLE 1
Characteristics of the various PLA2s

| | | | | SIZE | |
|---|---|---|---|---|---|
| | ORIGIN | | LOCATION | kDa | Ca2+ |
| SPLA2 | Group I | Mammal | Secreted | 13-15 | mM |
| | Cobra | | | | |
| | Pancreatic | | | | |
| | Group II | Mammal | Secreted | 13-15 | mM |
| | Vipers | | | | |
| | Synovial | | | | |
| | Group III | | Secreted | 16-18 | mM |
| | Bee | | | | |
| CPLA2 | Group IV | Mammal | Cytosolic | 85 | μM |
| | Ubiquitous | | | | |
| | Group V | Mammal | Cytosolic | 40 | no |
| | Myocardium | | | | |

These enzymes share the same substrate the phospholipids that they hydrolyze in SN2 position; they are 2-acyl hydrolases and release the fatty acid located in this position as well as a lyso-phospholipid.

However, these groups of enzymes differ by their function, their location, their regulation, the mechanism of their action, their sequence, their structure and their dependence upon divalent ions.

The first three groups were isolated as extracellular enzymes or secreted PLA2s (sPLA2s) and have a high number of disulfide bridges, a molecular mass of around 15 kDa and require, for their activity, a high concentration of calcium.

The classification of the PLA2s in one of these three groups is essentially made on the basis of the homologies of structure. Although the majority of the PLA2s be non-human enzymes, human secreted PLA2s do exist in the synovial fluid (group II) or in the human pancreas (group I).

The best characterised PLA2 is the secreted PLA2 of group II which originates from human synovial fluid. Group IV of the PLA2s contains only one intracellular enzyme called cytoplasmic PLA2 (cPLA2) of high molecular weight (85 kDa), which is specific to the phospholipids which are carriers of arachidonic acid; this cPLA2 requires concentrations of calcium which are compatible with an intra-cytoplasmic activation.

The enzyme is cytosolic and translocates to the membrane during cellular activation.

This enzyme does not possess a disulphide bridge and is activated by kinases of the family of PKCs and the family of MAPS.

Finally, a fifth group of PLA2, intra-cytoplasmic PLA2, has been described. This enzyme of an MW of 40 kDa is also specific to the phospholipids which carry arachidonic acid, and does not require calcium for its activation.

Very schematically, it would seem that the PLA2 of group IV be the enzyme which is preferentially used under physiological conditions and that the sPLA2 be synthesized and secreted in response to inflammatory stimuli, so as to produce mediators of the inflammation.

Nevertheless, this distinction is too schematic in fact, during the cellular activation occurring in an inflammatory reaction, the two PLA2s, sPLA2 and cPLA2, are induced and activated.

Various pieces of work treat modifications of the activity of the PLA2s secreted in psoriasis (Forster et al, Br. J. Dermatol. 1985, 112,135-147). The studies do not identify the specific forms of the PLA2.

### AIMS OF THE INVENTION

An aim of the invention is to provide an active principle in the field of inflammation, which is capable notably of reducing skin inflammation.

An aim of the invention is to solve the technical problem consisting in providing an active principle in the field of inflammation capable of inhibiting the enzymatic activity of phospholipase A2.

An aim of the invention is to solve the technical problem consisting in providing the use of these active principles in the cosmetic or dermo-pharmaceutical or pharmaceutical field, notably for the preparation of cosmetic compositions or dermo-pharmaceutical compositions or pharmaceutical compositions.

An aim of the invention is to solve the novel technical problem consisting in providing a method of testing the activity of a potentially active substance capable of inhibiting, in a significant manner, the enzymatic activity of the phospholipase A2.

The invention relates essentially to the phospholipases A2 of type I and/or of type II.

An aim of the invention is to solve the novel technical problem consisting of the use of this test method, for the research and the identification of a substance which is potentially active in the field of inflammation, which is based on the capacity of inhibition of the enzyme phospholipase A2.

An aim of the invention is to solve the technical problem consisting in providing an active principle, a cosmetic composition or derma-pharmaceutical composition or pharmaceutical composition containing the active principle, identified by said test method, notably for undergoing cares which enable reducing the signs of skin irritations, such as rashes or rednesses of the integument more or less linked to an external physical agent or to an inflammatory syndrome, xeroses or skin dryness, the loosening or loss of tone of the skin and blotchiness or the appearance of small burst vessels observed on very dry skins.

### DESCRIPTION OF THE INVENTION

The present invention enables solving the whole of the technical problems set forth above, particularly in a particularly unexpected manner.

Thus, the present invention provides a novel method of testing the activity of a potentially active substance capable of inhibiting, in a significant manner, the enzymatic activity of phospholipase A2.

The present invention also provides the use of this test method, for the research and the identification of a substance which is potentially active in the field of inflammation, which is based on the capacity of inhibition of the enzyme phospholipase A2.

According to a first aspect, the present invention provides a method of testing the activity of a potentially active substance to inhibit, in a significant manner, the enzymatic activity of phospholipase A2, preferably phospholipase A2 of type I or II, comprising

a) placing said potentially active substance in the presence with
the phospholipase A2;
a substrate which is a phospholipid, comprising at least one fatty acid in the form of an ester, the fatty acid is preferably a fatty acid having a long chain comprising between 15 and 22 carbon atoms, the substrate is more preferably unsaturated or poly-unsaturated, said substrate being capable of releasing at least one fatty acid during its hydrolysis;
   b) measuring the enzymatic activity of the phospholipase A2, notably comprising detecting the presence of said fatty acid released and eventually its quantitative determination.

This measurement of the enzymatic activity is made preferably by determination of the non-esterified fatty acids.

The inventors employ the term <<phospholipase A2>> in this part of the document with reference to the phospholipase A2 of type I and/or II.

By [symbol] to inhibit [symbol] , the inventors mean the fact that said active principle inhibits the phospholipase A2, so as to induce an enzymatic activity which is less than that induced without placing the phospholipase A2 in contact with the potentially active substance, all conditions of temperature, of contact time, and of operating conditions being identical, or comparable in other respects.

Advantageously, the inventors consider, within the context of the invention, that the selection of the potentially active molecules during screening can be made for inhibitions of the PLA2 activity which are qualified as very strong, when these inhibitions are greater than or equal to 50% of the reference activity, this reference activity being measured without the PLA2 being placed in contact with the potentially active substance, all conditions of temperature, of contact time, and of operating conditions being identical, or comparable in other respects.

In another advantageous embodiment, the test method is carried out with a phospholipase A2 of type I, notably for pre-selecting the potentially active substances, which are active at least in a significant manner, with reference to their inhibitory activity of phospholipase A2. The method is carried out again with the phospholipase A2 of type II, notably in order to confirm the potentially active substances which are capable of inhibiting, in a significant manner, the enzymatic activity of the phospholipase A2 of type I and/or of type II. This enables minimising the use of phospholipase A2 of type II which is not widely available and which is costly.

According to an advantageous embodiment, the enzyme phospholipase A2 of type I and/or of type II originates from bee (Apis mellifera) venom or originates from ox pancreas or originates from Streptomyces violaceoruber yeast, or originates from snake (Crotalus adarnanteus or Crotalus atrox or Crotalus Durissus or Naja mossambica mossambica) venom or originates from human or animal cell lysate or originates from human or animal biological fluid (synovial fluid), or originates from one of any possible mixture of the enzymes thus obtained.

According to an advantageous embodiment, the enzyme phospholipase A2 of type I originates from pig pancreas.

According to an advantageous embodiment, the enzyme phospholipase A2 of type II originates from human synovial fluid or originates from Crotalus adamanteus snake venom.

According to an advantageous embodiment, the substrate is of phospholipid nature comprising, in type 2 nucleophilic substitution (SN2) position, at least one fatty acid having a long chain, preferably a long chain of between C15 and C22 carbon atoms, this fatty acid being more preferably unsaturated or poly-unsaturated.

According to an advantageous embodiment, the substrate is selected from at least one ester derivative of arachidonic acid, preferably the substrate is β-arachidonoyl-γ-palmitoyl L-α-phosphatidylcholine.

According to an advantageous embodiment, the method comprises placing in contact with a cofactor of the phospholipase A2.

The concentrations of cofactor are preferably between 0.0001% and 10%. Advantageously, the cofactor is a bivalent ion. Even more advantageously, the cofactor is the specific cofactor, which is calcium.

According to an advantageous embodiment, the method comprises the placing in contact with an agent of dissolution. The concentrations of dissolution agent are preferably between 0.001% and 10%. Advantageously, the dissolution agent is sodium deoxycholate.

According to a second aspect, the present invention relates to the use of a method of testing as defined above, or in the following description, for identifying at least one active principle which is capable of inhibiting, in a significant manner, the enzymatic activity of phospholipase A2, notably of phospholipase A2 of type I and/or II.

Advantageously, the enzymatic activity of the phospholipase A2 is inhibited from the moment when the phospholipase A2 activity, measured in the presence of the active, is less than the activity measured without placing the phospholipase A2 in contact with said potentially active substance, all conditions of temperature, of contact time, and of operating conditions being identical, or comparable in other respects,

According to a third aspect, the present invention relates to an active principle which is capable of inhibiting the enzymatic activity of phospholipase A2, notably of phospholipase A2 of type I and/or H, the activity of said phospholipase A2 being measured in executing the placing of said phospholipase A2 in contact with
said active principle
a substrate which is a phospholipid, comprising at least one fatty acid in the form of an ester, the fatty acid is preferably a fatty acid having a long chain of 15 to 22 carbon atoms, the fatty acid is more preferably unsaturated or poly-unsaturated, said substrate being capable of releasing at least one fatty acid during its hydrolysis.

The different embodiments of the test method which are described above can be implemented for identifying and/or for selecting an active principle as described above. That is to say, notably, that:

According to an advantageous embodiment, the enzyme phospholipase A2 of type I and/or of type II originates from bee (Apis mellifera) venom or originates from ox pancreas or originates from Streptomyces violaceoruber yeast, or originates from snake (Crotalus adamanteus or Crotalus atrox or Crotalus Durissus or Naja mossambica mossambica) venom or originates from human or animal cell lysate or originates from human or animal biological fluid (synovial fluid), or originates from one of any mixture possible of the enzymes thus obtained.

According to an advantageous embodiment, the enzyme phospholipase A2 of type I originates from pig pancreas.

According to an advantageous embodiment, the enzyme phospholipase A2 of type II originates from human synovial fluid or originates from Crotalus adamanteus snake venom.

According to an advantageous embodiment, the substrate is of phospholipid nature comprising, in type 2 nucleophilic substitution (SN2) position, at least one fatty acid having a long chain, preferably a long chain of between C15 and C22 carbon atoms, this fatty acid being more preferably unsaturated or poly-unsaturated.

According to an advantageous embodiment, the substrate is selected from at least one ester derivative of arachidonic acid, preferably the substrate is β-arachidonoyky-palmitoyl L-α-phosphatidylcholine.

According to an advantageous embodiment, the method comprises placing in contact with a cofactor of the phospholipase A2.

The concentrations of cofactor are preferably between 0.0001% and 10%. Advantageously, the cofactor is a bivalent ion. Even more advantageously, the cofactor is the specific cofactor, which is calcium.

According to an advantageous embodiment, the method comprises the placing in contact with an agent of dissolution. The concentrations of dissolution agent are preferably between 0.001% and 10%. Advantageously, the dissolution agent is sodium deoxycholate.

According to a fourth aspect, the present invention relates to an active principle which is capable of inhibiting the enzymatic activity of phospholipase A2, notably of phospholipase A2 of type I and/or II, said active principle being identified by the test method as defined above.

According to a fifth aspect, the present invention relates to an active principle having an anti-inflammatory and/or anti-pain and/or anti-irritation and/or anti-prickling and/or anti-burn and/or anti-itching and/or anti-rash and/or anti-xerosis and/or anti-blotchiness and/or anti-skin tissue-loosening effect, characterised in that it is selected from an extract of grape seeds, an extract of Pueraria lobata, an extract of Pneumus boldus (boldo), an extract of arnica, an extract of lemon, an extract of sunflower, an extract of camomile, zinc gluconate, an extract of guarana and an extract of liana (Uncaria tomentosa), or one of the combinations resulting from the combination of at least two of the active principles listed, the plant extracts being preferably used at a concentration of between 0.1 and 30% (w/w) by weight of the final product.

According to a sixth aspect, the present invention relates to an active principle which is capable of inhibiting, in a significant manner, the enzymatic activity of phospholipase A2, notably of phospholipase A2 of type I and/or II, characterised in that it is selected from an extract of grape seeds, an extract of Pueraria lobata, an extract of Pneumus boldus (boldo), an extract of arnica, an extract of lemon, an extract of sunflower, an extract of camomile, zinc gluconate, an extract of guarana and an extract of liana (Uncaria tomentosa), or one of the combinations resulting from the combination of at least two of the active principles listed, the plant extracts being preferably used at a concentration of between 0.1 and 30% (w/w) by weight of the final product.

According to a seventh aspect, the present invention relates to a plant extract of Pueraria Lobata root, preferably extracted at a concentration of between 0.1% and 20% by weight, preferably at a concentration of about 5% (e.g. about 5 g qsp 100 g of solvent), in an aqueous solvent containing an alcohol/glycol such as, for example, butylene glycol and/or ethanol, e.g. at a concentration of between 0% and 80%, preferably at a concentration of about 25% of butylene glycol, and eventually a preservative such as methyl paraben at a concentration of between 0.01% and 0.5%, preferably at a concentration of about 0.1% (w/w). The extract called [symbol] Extract 1 [symbol] is made from an aqueous extraction only.

According to an eighth aspect, the present invention relates to a plant extract of grape seeds, made from grape seeds, preferably extracts at a concentration of between 0.1% and 20% by weight, preferably at a concentration of about 2% (e.g. about 2 g qsp 100 g of solvent), in an aqueous solvent containing an alcohol/glycol such as, for example, butylene glycol and/or ethanol, e.g. at a concentration of between 0% and 80%, preferably at a concentration of about 25% of butylene glycol, and eventually a preservative such as methyl paraben at a concentration of between 0.01% and 0.5%, preferably at a concentration of about 0.1% (w/w). The extract called [symbol] Extract 2 [symbol] is made from an aqueous extraction only.

According to a ninth aspect, the present invention relates to a plant extract of boldo, made from boldo leaves, which are preferably extracted at a concentration of between 0.1% and 20% by weight, preferably at a concentration of about 2% (e.g. about 2 g qsp 100 g of solvent), in an aqueous solvent containing an alcohol/glycol such as, for example, butylene glycol and/or ethanol, e.g. at a concentration of between 0% and 80%, preferably at a concentration of about 25% of butylene glycol, and eventually a preservative such as methyl paraben at a concentration of between 0.01% and 0.5%, preferably at a concentration of about 0.1% (w/w).

According to a tenth aspect, the present invention relates to a plant extract of arnica, made from the arnica plant, preferably extracted at a concentration of between 0.1% and 20% by weight, preferably at a concentration of about 2% (e.g. about 2 g qsp 100 g of solvent), in an aqueous solvent containing an alcohol/glycol such as, for example, butyiene glycol and/or ethanol, e.g. at a concentration of between 0% and 80%, preferably at a concentration of about 25% of butylene glycol, and eventually a preservative such as methyl paraben at a concentration of between 0.01% and 0.5%, preferably at a concentration of about 0.1% (w/w).

According to an eleventh aspect, the present invention relates to the use of at least one active principle as defined above or in the following description, and/or of an extract as defined above or in the following description, for preparing a composition, notably a cosmetic composition, used with the aim of reducing the irritations and/or the pricklings and/or the itchings and/or of limiting the superficial observations of blotchiness and/or the appearance of small burst vessels and/or the loosening of the skin tissues and/or the loss of tone of the skin and/or the dryness of the skin.

According to a twelfth aspect, the present invention relates to the use of at least one active principle as defined above and/or of an extract as defined above or in the following description, for preparing a composition, notably of a pharmaceutical composition, used with the aim of reducing the inflammations and/or the pains and/or the burns and/or the rashes and/or the rednesses of the integument more or less linked to an external physical agent or to an inflammatory syndrome and/or the xeroses.

According to a thirteenth aspect, the present invention relates to the use of at least one active principle as defined above or in the following description and/or of an extract as defined above or in the following description, for preparing a cosmetic composition, used with the aim of inhibiting the enzymatic activity of phospholipase A2, notably of phospholipase A2 of type I and/or II.

According to a fourteenth aspect, the present invention relates to the use of at least one active principle as defined above or in the following description and/or of an extract as defined above or in the following description, for preparing a pharmaceutical composition, used with the aim of inhibiting the enzymatic activity of phospholipase A2, notably of phospholipase A2 of type I and/or II.

According to a fifteenth aspect, the present invention relates to a cosmetic composition comprising at least one active principle as defined above or in the following description and/or of an extract as defined above or in the following description.

According to a sixteenth aspect, the present invention relates to a pharmaceutical composition comprising at least one active principle as defined above or in the following description and/or of an extract as defined above or in the following description.

Preferably, the concentration of the active principle according to the present invention is between 0.01% and 30% by weight of the total composition.

The active principles can advantageously be combined between themselves for treating several symptoms or for more effectively treating a same symptom.

According to a seventeenth aspect, the present invention relates to a method of cosmetic care comprising topically applying a cosmetic composition as defined above, on the areas of the skin of a person in need thereof.

Advantageously, the cosmetic care method relates to the cares of the irritations and/or the pricklings and/or the itchings and/or cares in order to limit or to do away with the superficial observations of blotchiness and/or the appearance of small burst vessels and/or the loosening of the skin tissues and/or the loss of tone of the skin tissues and/or the dryness of the skin tissues.

Advantageously, these skin tissues comprise skin.

### DETAILED DESCRIPTION OF THE INVENTION

The study of the inhibition of phospholipase A2 (PLA2) is carried out in an acellular in vitro model which aims to be a reflection, near as possible, of the situation encountered in vivo.

The PLA2s of type I and of type II are used in vitro, in a model comprising:
1) a substrate selected from the ester derivatives of arachidonic acid (such as β-arachidonoyl γ-palmitoyl L-α-phosphatidylcholine, which is a phospholipid containing arachidonic acid in SN2 position, site hydrolyzed by the phospholipase A2) which specifically is the fatty add involved in the synthesis of the mediators of the inflammation,
2) a bivalent ion, playing the role of catalyst (such as calcium),
3) an activator, which is indispensable for the activity of the enzyme, playing the role of solubilizer of the substrate in the reaction medium and enabling promoting the enzyme-substrate interaction (preferably sodium deoxycholate).

This reaction mixture is placed in the presence of various substances which are potentially active, the activity of which of inhibition of the PLA2 is to be tested, and the content of free fatty acids after the test can be evaluated in various ways (vapor phase chromatography, HPLC, colorimetric determination, etc. . . ), and this enables selecting the best inhibitors.

The PLA2 which is preferably used in the study model originates from pig pancreas (enzyme of type I), for reasons of availability and of cost, being understood that
the homologies of sequence between the PLA2 of type I and the human equivalent PLA2 of type II (Tatina A et al, Blast 2 sequences-a new tool for comparing protein and nucleotide sequences, FEMS Microbil Lett, 174, 247-250 (1999)) are more than 54%,
parallel studies have enabled the inventors to show that these two enzymes have spectra of activity which are relatively similar according to the inhibitors which are presented to them.

The PLA2 is placed in the presence of a substrate, e.g. β-arachidonoyl γ-palmitoyl L-α-phosphatidylcholine, which is a phospholipid containing arachidonic acid in SN2 position, site hydrolyzed by the phospholipase A2, which is specifically the fatty acid involved in the synthesis of the mediators of the inflammation,

In parallel, controls for the inhibition of the phospholipase A2 can be done, which can, for example, be
aristolochic acid (8-methoxy-6-nitrophenanthro(3,4-a)-1,3-dioxole-5-carboxylic acid), which is a major constituent isolated from various Aristolochia plant species, is used in traditional medicine for neutralising snake venoms, notably from Naja naja atra and Bungarus multicinctus.

It has been demonstrated that aristolochic add specifically inhibited in vitro the enzymatic activity and the edema-inducing activity of the PLA2 originating from snake venoms (Vishwanath B. S. Edema-Inducing activity of phospholipase A2 purified from human synovial fluid and inhibition by aristolochic acid, Inflammation, Vol 12, N°6, 549-561, 1988, Sannanaik Vishwanath B, Interaction of aristolochic acid with vipera Russelli phospholipase A2: its effect on enzymatic and pathological activities Toxicom, 25, 929-937, 1987; Moreno J J, Effect of aristolochic acid on arachidonic acid cascade and in vivo models of inflammation, Immunopharmacology, 26, 1-9, 1993).
p-bromophenacyl bromide, which is a specific inhibitor of the secreted PLA2s (Mao-Qiang M et al, Secretory phospholipase A2 activity is required for permeability barrier homeostasis, J. Invest. Derm., 106, 1996, 57-63).

To the reaction medium are also added
a catalyst, which is preferably calcium.
An activator, which is indispensable for the activity of the enzyme, which is, for example, sodium deoxycholate. This activator enables increasing the solubility of the substrate in the reaction medium and therefore promotes the enzyme-substrate interaction.
   The study of the inhibition of the PLA2 is preferably made in two phases.

The enzyme in the presence of its cofactor is incubated for a determined period of time (e.g. about 15 minutes) with the inhibitor, and then a second incubation is carried out for (e.g. about 20 minutes) in the presence of the substrate which is preferably β-arachidonoyl γ-palmitoyl L-α-phosphatidylcholine and of the activator, which is preferably sodium deoxycholate.

At the end of this incubation, a determination of the non-esterified fatty acids is made on the reaction medium by an enzymatic technique that it is for example possible to follow by colorimetry at a defined wavelength.

In parallel, a control corresponding to the activity of the phospholipase A2 in the absence of inhibitor is carried out. The use of an active which is capable of significantly inhibiting the enzymatic activity will manifest itself by a decrease of the optical density at the defined wavelength, i.e. by a lowering of the fatty acids released in the medium with respect to the control.

In this manner, it has been possible to carry out the screening of potentially active substances which are capable of inhibiting the enzyme PLA2.

By this technique, a screening over about a hundred molecules has been made, so as to select, from various families of potential active principles, namely plant extracts, algae, polysaccharides or proteins, those having a strong inhibitory activity of PLA2.

The products arising from the screening and therefore possessing an inhibitory activity of the. PLA2 of type I, are then evaluated through a similar test method which makes use of the placing of the potentially active substance in contact with a PLA2 of type II, originating from Crotalus Adamanteus (snake venom), enzyme of choice found in the skin tissues during inflammatory processes.

This test with the PLA2 of type II is in general made after that which makes use of the PLA2 of type I, for reasons of availability and of cost, as set forth above. It suffices thus for this reason to pre-select the potentially active substances with the test method making use of the PLA2 of type I and then to confirm or to refute the activity of the pre-selected substances with the test method which makes use of the PLA2 of type II.

Thus, specific extracts have been selected on the basis of their efficiency: extracts of grape seeds, extracts of Pueraria Lobata, extracts of boldo (Pneumus boldus), extracts of lemon, extracts of sunflower, extracts of camomile, zinc gluconate, extracts of guarana, extracts of liana (Uncaria tomentosa), extracts of arnica.

### DESCRIPTIONS OF THE FIGURES

FIG. 1 shows the results of anti-PLA2 activity test for various concentrations of Extract 1, the concentration of Extract 1 being expressed in percentage on the abscissa; and the level of PLA2 inhibition being expressed in percentage on the ordinate, and this for Example 2 relating to the extract of Pueraria Lobata, or Extract 1;

FIG. 2 represents, in a similar way to FIG. 1, a comparison of anti-PLA2 activity between a PLA2 of type I originating from Extract 1 of Pueraria Lobata, and a PLA2 of type II originating from Crotalus Adamanteus, the subject of Table IV;

FIG. 3 represents the results obtained of anti-PLA2 activity for various concentrations of Extract 2, extract of grape seeds, according to Example 3, with the concentration of Extract 2 in percentage on the abscissa and the PLA2 inhibition in percentage on the ordinate;

FIG. 4 is a curve similar to FIG. 2 for Extract 2, comparing the PLA2 of type I with the PLA2 of type II;

FIG. 5 represents the distribution of the number of volunteers, whose signs of skin irritation have increased, have not been modified or have reduced after 28 days of use of a placebo formulation or a formulation containing 3% of Extract 1 of Pueraria according to the in vivo study of Example 6;

FIG. 6 represents the improvement of the signs of skin irritation after 28 days of use of a placebo formulation or a formulation containing 3% of an Extract 1 of Pueraria, according to the in vivo study of Example 6;

FIG. 7 represents the results of reducing of the intensity of the signs of skin irritations in percentage, comparing a placebo formulation with a formulation containing 3 ° A) of Extract 1, according to the in vivo study of Example 6;

FIG. 8 represents the number of volunteers, in percentage of the numbers having a softened skin or a skin made supple, between a placebo formulation and a formulation containing 3% of Extract 1 within the context of the in vivo study on human volunteer of Example 6;

FIG. 9 represents, in a manner similar to FIG. 8, the number of volunteers, in percentage of the numbers desiring to carry out the treatment or having a pressure to buy between a placebo formulation and a formulation containing 3% of Extract 1.

In the Examples, any characteristic which appears to be novel with respect to any state of the art makes up an integral part of the present invention and the protection is sought in its function and its generality.

Furthermore, in the description and the claims, all percentages are given by weight, the temperature is in degrees Celsius, the pressure is atmospheric pressure, unless indicated otherwise.

### EXAMPLE 1

### Of the Invention Making use of the Screening of Actives

The PLA2 in aqueous solution (35 Units/ml), is placed in the presence of 3 mM β-arachidonoyl γ-palmitoyl L-α-phosphatidylcholine, which is a phospholipid containing, in SN2 position, site hydrolyzed by the phospholipase A2, arachidonic add, which is specifically an important fatty acid involved in the synthesis of the mediators of inflammation,

To the reaction medium are also added
calcium (cofactor) 0.9 mM
sodium deoxycholate (activator) 1.6 mM
   The study of the inhibition of the PLA2 is made in two phases

a) the enzyme in the presence of the cofactor (calcium) is incubated for 15 minutes at ambient temperature (20° C.) with the inhibitor;

b) then, a second incubation of 20 minutes is made in the presence of β-arachidonoyl γ-palmitoyl L-α-phosphatidylcholine (3 mM) and sodium deoxycholate (1.6 mM).

At the end of this incubation, a determination of the free fatty acids, thus non-esterified free fatty acids, is made on the reaction medium by a classical enzymatic technique which is well-known to the person skilled in the art, that it is possible to follow by colorimetry, e.g. here at 550 nm,

The controls used in accordance with the invention are
aristolochic acid (8-methoxy-6-nitrophenanthro(3,4-d)-1,3-dioxole-5-carboxylic acid),
p-bromophenacyl bromide, which are specific inhibitors of the secreted PLA2s.

These molecules have enabled obtaining the following results:

| | Inhibition (%) | | |
|---|---|---|---|
| | Non-diluted | | |
| | product | | |
| | (used at the | Product diluted | Product diluted |
| | concentrations | at 1/100 in | at 1/1000 in |
| Inhibitors | cited in | demineralized | demineralized |
| (initial concentrations) | column 1) | water | water |
| Aristolochic acid | 96.2% ± 1.04 | 10.00% ± 3.4 | 0.25% ± 0.5 |
| (2 mM) | | | |
| p-bromophenacyl | 29.9% ± 8.3 | 4.5% ± 2.3 | — |
| bromide (10 mM) | | | |
| nordihydroguaiaretic | 97.3% ± 13.2 | 8.8% ± 0.8 | — |
| acid (5 mM) | | | |

The potentially active substances tested and the results are listed in Table II below

TABLE II
Results of the tests of screening for the products made and
marketed by Coletica: the results are expressed in percentage inhibition
with respect to the control.

| | Inhibition | Inhibition by substance |
|---|---|---|
| | by substance | diluted at 1% in |
| Potentially active substance | tested pure | demineralized water |
| tested | (%) | (%) |
| Extract of Pumpkin | 1.3 | 1.5 |
| Extract of Liana | 55.3 | 0 |
| Extract of lucerne | 19.4 | 1.7 |
| Extract of cress | 0 | 0 |
| Extract of lemon | 23.5 | 1.4 |
| Extract of bilberry | 9.2 | 2.2 |
| Extract of mulberry | 0 | 0 |
| Extract of Pueraria | 90.2 | 23.9 |
| Extract of sunflower | 25.5 | 0 |
| Extract of grape seed | 79.82 | 29.7 |
| Extract of St. John's wort | 35.7 | 0 |
| Extract of Shiitake | 0 | 0 |
| Extract of alga | 1.7 | 0 |
| Zinc gluconate | 50 | 12.6 |
| (5% aqueous solution w/w) | | |
| Magnesium gluconate | 3.4 | 0 |
| (5% aqueous solution w/w) | | |
| Extract of mushroom | 0 | 0 |
| Extract of liquorice | 5.8 | 0 |
| Flour of lupin (extraction of 5 g | 2.6 | 0 |
| in 95 g of water) | | |
| Extract of camomile | 41.3 | 0 |
| Extract of vanilla | 0 | 0 |
| Extract of Guarana | 59.1 | 1.1 |
| Extract of saxifrage | 2.6 | 0 |
| Extract of Lentinus edodes | 0.9 | 0 |
| Extract of Peumus boldus | 80.2 | 72.4 |
| Extract of arnica | 79.8 | 69.2 |
| Coletica exopolysaccharide | 24.2 | 0 |
| made by fermentation (1% | | |
| w/w in demineralized water) | | |

The extracts of pumpkin, of lucerne, of cress, of lemon, of mulberry, of sunflower, of St. John's wort, of liquorice, of camomile, of vanilla, of Guarana, of saxifrage, of Lentinus edodes and of Pneumus boldus are made in the following way: a soaking of the leaf at 5% (w/w) in water, or of the entire plant at 5% (w/w) in water or of the fruits at 5% (w/w) in water, is made for 1 night at 4° C. Then, the suspension obtained is filtered over 0.45 μm. The determination of the inhibitory activity of PLA2 is made directly on the filtrate obtained.

The extracts of Liana, of bilberry, of Pueraria Lobata and of grape seeds are made after grinding of the roots or entire plants or of the fruit, and then alcoholic extraction at 5% (w/w) in 70% ethanol.

A decoction is made by heating the mixture at 60° C. for 1 hour. The supernatant is then filtered. A second decoction is made from the plug obtained in the same proportion at 5% (w/w) in 70% ethanol. The alcohol of the two supernatants obtained is evaporated off with a rotary evaporator, and the plug is then dried by lyophilization.

The dry product obtained is re-dissolved at 5% (w/w) in a mixture made up of 69.6% water (w/w), butylene glycol (25%), methyl paraben (0.1%).

The solution of “flour of lupin” is obtained from a dissolution of a mixture of lupin protein and polysaccharide at 5%(w/w) in water.

At the end of this study enabling to research for active substances, specific extracts have been selected on the basis of their effectiveness the extract of grape seeds, the extracts of pueraria lobata, the extracts of boldo (Pneumus boldus), the extracts of lemon, the extracts of acacia, the extracts of sunflower, the extracts of camomile, zinc gluconate, the extracts of guarana, the extracts of liana (urticaria tomentosa).

### EXAMPLE 2

### Of the Present Invention

### 1—Extract of Pueraria Lobata or Extract 1

### A—Generalities

Pueraria lobata (Kudzu, Ge-gen) is an original plant, which possesses voluble stems, such as the vine shoots of a vine, which enable it to attach itself to netting or to trees.

This plant, which originates from China and Japan, where its root is used in cooking as starch, is known in Chinese medicine since the VIth Century BC for numerous properties, one of which has been the subject of recent pieces of research by the Americans: that of promoting overcoming drug addiction. The root of Pueraria Lobata contains 3 flavonoids: puerarin, dadzein and dadzine. This root is consumed regularly as a cure, since it leads to a decrease in the consumption of alcohol and a greatly-reduced cigarette dependence (Shebek, J et al, Journal of alternative and complementary medicine, 45-48, 2000).

### B—Composition of Extract 1

### Extract 1 is Made After Grinding the Roots and the 5% Alcohol Extraction in 70% Ethanol.

A decoction is made by heating the mixture at 60° C. for 1 hour and then the supernatant is filtered. A second decoction is made from the plug obtained in the same proportion at 5% (w/w) in 70% ethanol. The alcohol of the two supernatants obtained is evaporated of with a rotary evaporator and then the plug is dried by lyophilization.

The dry product obtained is re-dissolved at 5% (w/w) in a mixture made up of 69.6% water (w/w), butylene glycol (25%), methyl paraben (0.1%).

C—Anti-PLA2 activity

### C1—Determination of the Free Fatty Acids (Non-Esterified)

A study of the dose dependence of the effects of the aqueous plant extract (called “Extract 1”) was made so as to evaluate the specificity of action of the product selected towards the PLA2 of type Z originating from pig pancreas.

The anti-PLA2 activity of increasing concentrations of the product selected was measured over three different batches of starting material. Each determination was made in triplicate,

The results obtained are listed in Table III below

TABLE III
Concentrations of use of Extract 1(%)

| | 10 | 5 | 3 | 2 | 1 | 0.1 |
|---|---|---|---|---|---|---|
| Inhibition (%) | 88.62 | 77.31 | 58.73 | 44.2 | 21.66 | 0.38 |
| Standard | 0.36 | 0.80 | 2.33 | 1.78 | 1.4 | 0.36 |
| deviation (%) | | | | | | |

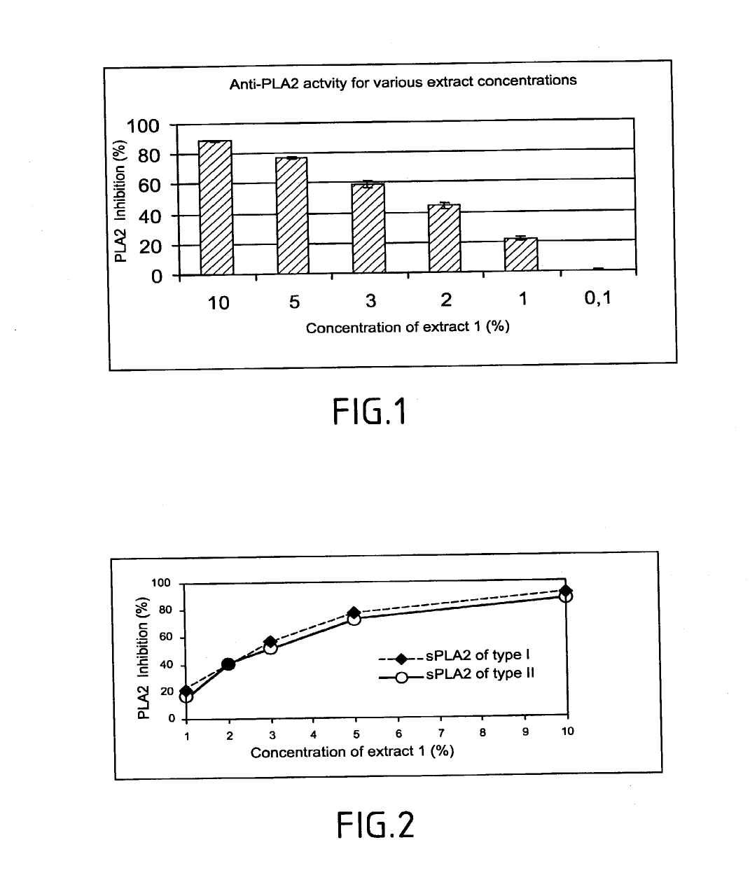

The results are expressed in percentage inhibition with respect to control and are the subject of FIG. 1,

In FIG. 1, the results show that the inhibitor effect of the product selected towards the PLA2 is dose-related. This result is in favor of a specificity of action of this compound for the parameter studied.

### C2—Activity Measured on a PLA2 of Type II

A dose effect curve is made on a PLA2 of type II originating from Crotalus adamanteus so as to validate the results obtained on the PLA2 of type I used in our screening model.

TABLE IV

| Concentration | | | | | |
|---|---|---|---|---|---|
| of use (%) | 10% | 5% | 3% | 2% | 1% |
| sPLA2 type I | 91.3 | 77.5 | 56.6 | 40.7 | 21.6 |
| average | | | | | |
| Standard | 0.36 | 0.79 | 3.44 | 2.19 | 2.65 |
| deviation | | | | | |
| sPLA2 type II | 87.3 | 72.3 | 51.6 | 40.1 | 16.9 |
| Mean | | | | | |
| Standard | 1.49 | 0.52 | 0.99 | 1.52 | 1.85 |
| deviation | | | | | |

The results are expressed in percentage inhibition with respect to control and are the subject of FIG. 2.

In FIG. 2, the results presented here show that the inhibitory activity of the product selected towards the PLA2 of type I or of type II are equivalent. The product selected is thus indeed capable of inhibiting the form of PLA2 encountered in the skin tissues during inflammation and from this fact constitutes a tool of choice in order to come to the aid of sensitive skins.

### EXAMPLE 3

### Of the Present Invention

Extract from Grape Seeds (Extract 2)

Extract 2 is made after harvest of the seeds and alcohol extraction at 5% (w/w) in 70% ethanol.

A decoction is made in heating the mixture at 60° C. for 1 hour and then the supernatant is filtered. A second decoction is made from the plug obtained in the same proportion at 5% (w/w) in 70% ethanol. The alcohol of the 2 supernatants obtained is evaporated off with a rotary evaporator and then the plug is dried by lyophilization.

The dry product obtained is re-dissolved at 2% (w/w) in a mixture made up of 72.6% water (w/w), butylene glycol (25%), methyl paraben (0.1%).

### Anti-PLA2 Activity

A study of the dose dependence of the effects of this substance was made so as to evaluate the specificity of action of the product selected towards the PLA2.

The anti-PLA2 activity of increasing concentrations of the product selected was evaluated over 3 different batches of starting material. Each determination was made in triplicate.

The results obtained are listed in Table V below

TABLE V

| Concentration | | | | | |
|---|---|---|---|---|---|
| of use (%) | 5 | 3 | 2 | 1 | 0.1 |
| Inhibition (%) | 71.75 | 59.41 | 47.80 | 32.78 | 3.40 |
| Standard deviation | 3.95 | 2.62 | 1.98 | 3.42 | 1.52 |
| (%) | | | | | |

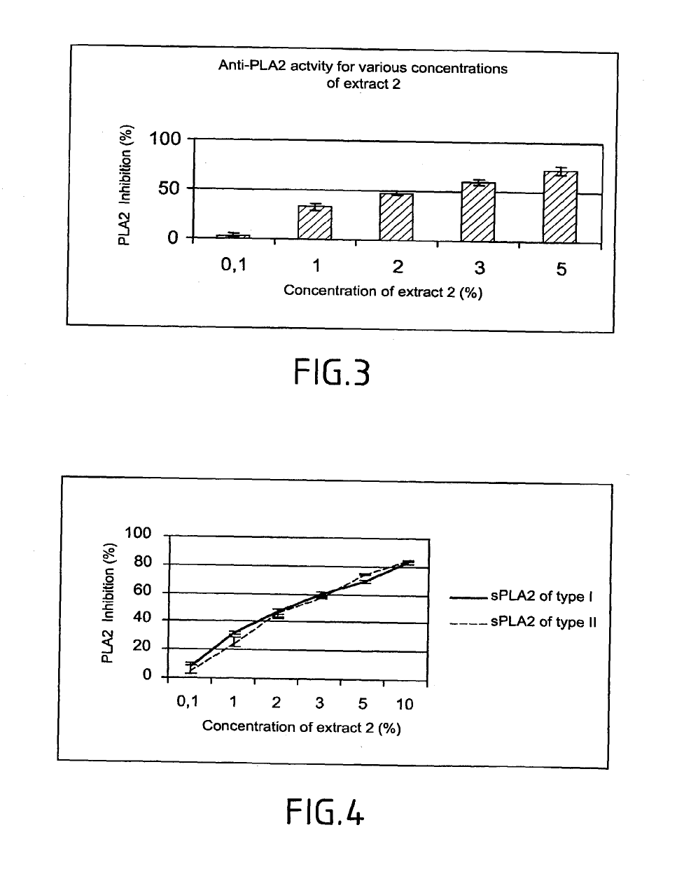

The results are expressed in percentage inhibition with respect to control and are the subject of FIG. 3.

In FIG. 3, the results show that the inhibitor effect of the product selected towards the PLA2 is dose-related. This result is in favor of a specificity of action of this compound for the parameter studied.

Activity Measured on a PLA2 of type II

A dose effect curve is made on a PLA2 of type II so as to validate the results obtained on the PLA2 of type I used in our screening model.

TABLE VI

| | 10% | 5% | 3% | 2% | 1% | 0.1% |
|---|---|---|---|---|---|---|
| sPLA2 type I | 84.1 | 69.5 | 60.1 | 46.2 | 31.9 | 66 |
| Standard | 0.89 | 1.06 | 1.21 | 2.49 | 0.77 | 3.98 |
| deviation | | | | | | |
| sPLA2 type II | 82.3 | 74.1 | 57.1 | 43.7 | 25.6 | 5.6 |
| Standard | 0.99 | 0.49 | 0.47 | 1.58 | 3.2 | 2.93 |
| deviation | | | | | | |

The results are expressed in percentage inhibition with respect to control and are the subject of FIG. 4.

In FIG. 4, the results presented here show that the inhibitory activities of the product selected towards the PLA2 of type I or of type II are equivalent. The product selected is thus indeed capable of inhibiting the form of PLA2 encountered in the skin tissues during inflammation and from this fact constitutes a tool of choice in order to come to the aid of sensitive skins.

### EXAMPLE 4

### Of the Present Invention

### Activity of the Anti-Inflammatories

The steroidal and non-steroidal anti-inflammatories which are classically used in pharmacy are tested in our study model so as to compare their activity with respect to the effectiveness demonstrated of the products selected above.

The results are expressed in percentage inhibition with respect to control

TABLE VII

| Non-steroid | | |
|---|---|---|
| ant-inflammatories | 10 mM | 1 mM |
| Diclofenac | 29.8 | 8 |
| Indomethacin | 25.7 | 0 |
| Ibuprofen | 40.3 | 6.9 |
| Aspirin | 7 | 3.5 |
| Steroidal anti-inflammatories | 10 mM | |
| Cortisone | 0 | |
| Hydrocortisone | 4.5 | |
| Prednisolone | 6.3 | |

The results obtained, in our study model, show that the anti-inflammatories classically studied have an anti-PLA2 activity which is less than the actives selected and described above, used at 3% in a formula.

### EXAMPLE 5

### Of the Present Invention

### Identification of the Isoflavones Present in Extract of Puerada

Pueraria Lobata is a plant which is known for its content of isoflavones such as puerarin, dadzein, dadzine and genistein. (Kaufman P, et al, 1997). These molecules have been determined in the product selected and described above by an HPLC technique.

The contents of puerarin, dadzin and dadzeine are mentioned in Table VIII below:

TABLE VIII

| Isoflavones | Content (%) |
|---|---|
| puerarin | 1.5 |
| daidzine | 0.45 |
| dadzeine | 0.06 |
| genistein | <0.005 |

It was not possible to make a determination of genistein in the product selected and described above due to its very low concentration, calculated to be less than 0.005%.

However, a commercial 1% genistein solution was evaluated in our model of inhibition of the PLA2, and the result obtained (28.79%), and this shows that this isoflavone is not responsible for the activity of the extract selected insofar as the genisteine content of this product is less than 0.005%.

The anti-PLA2 activity of the mixture of the 3 isoflavones at the concentrations found in the extract is evaluated in the model of inhibition developed.

The results show that the isoflavones identified in the extract of pueraria are not responsible for the inhibitory activity of the PLA2.

### EXAMPLE 6

### Of the Present Invention

### In Vivo Study on Human Volunteers

A study on human volunteers is made so as to test the effectiveness of a formulation, containing 3% of Extract 1, with respect to signs of skin irritations observed during inflammation of the skin.

### 1—Protocol of the Study

50 volunteers used a placebo formulation for 28 days. 50 other volunteers used a formulation containing 3% of an extract 1 for the same period of time.

Each group of 50 volunteers was divided into two sub-groups of 25 volunteers. One sub-group was made up of volunteers having a reactive skin (<<sensitive skin>> sub-group, SS), the other was made up of volunteers estimating to have a reactive skin (<<estimated sensitive skin>> sub-group, ESS). The inclusion of the volunteers in each sub-group was made with the aid of a clinical questionnaire validated for two years by the laboratory having led the study.

N.B. The volunteers of the two sub-groups (SS+ESS) could present signs of skin irritation.

Before and after the 28 days of treatment by one or the other of the formulations (placebo formulation or formulation containing Extract 1):

the signs of skin irritation presented by the volunteers were <<rated>> by a doctor.

The various signs observed were the following:
Rash: More or less localized blotchiness of the integument linked to an external physical agent or to an inflammatory syndrome.
Xerosis: Medical term used to define the dryness of the skin but with a connotation of intensity. The term [symbol] xerosis [symbol] is used when it is desired to define dryness which is more than moderate.
Loosening: tone of the skin analyzed clinically by capacity of recovery of the skin subjected to constraints.
Blotchiness: Small burst vessels (telangiectasiae) in the form of stars, of copper-rose color occurring on dry skins on the face.
   At the end of the study, the volunteers responded to a questionnaire of evaluation of the products.

### Results

### Overall Analysis

If no distinction is made between the SS and ESS sub-groups and that the distribution of the volunteers is examined whose signs of skin irritation have increased, have not been modified or have reduced during the study, it can be observed

For the volunteers having used the placebo formulation:
4 saw their signs of skin irritation increased
23 did not see their signs of skin irritation being modified
23 saw their signs of skin irritation reduce
   For the volunteers having used the formulation containing 3% of an Extract of 1:
0 saw their signs of skin irritation increased
19 did not see their signs of skin irritation being modified
31 saw their signs of skin irritation reduce.

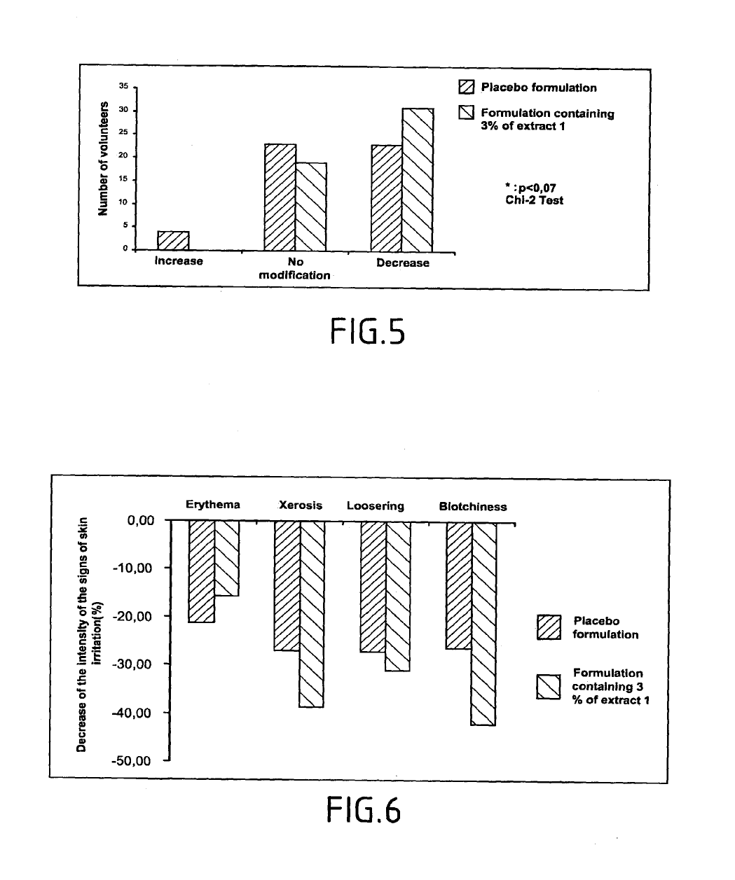

These results, which are presented in FIG. 5 clearly show that the formulation which contains Extract 1 proposes an improvement of the clinical signs of skin irritation which is greater than that proposed by the placebo formulation (p<0.07, Chi-2 test).

In FIG. 5, it is to be noted that the distribution of the number of volunteers whose signs of skin irritation have increased, have not been modified or have reduced after 28 days of use of a placebo formulation or a formulation containing 3% of extract of pueraria.

### Item by Item Analysis

If no distinction is made between the SS and ESS sub-groups, the two formulations (placebo formulation and formulation containing Extract 1) are capable of significantly improving most of the clinical signs of skin irritation encountered in the panelists (FIG. 6).

In FIG. 6, an improvement is observed of the signs of skin irritation after 28 days of use of a placebo formulation or a formulation containing 3% of an Extract 1: Overall Analysis without distinction between the SS and ESS sub-groups.

The following comments can be made in relation to FIG. 6:
ns: no significant difference between D0 and D28
*: significant difference between D0 and D28 (p<0.05; Wilcoxon's Paired Signed Rank Test)
**: significant difference between D0 and D28 (p<0.001; Wilcoxon's Paired Signed Rank Test)
***: significant difference between D0 and D28 (p<0.001; Wilcoxon's Paired Signed Rank Test).
   Percentages of decrease of intensity of the clinical signs of skin
irritation:
Placebo formulation:
Rash −21.28+/−48.06%
Xerosis −27.03+/−72.23%
Loosening −27.12+/−43.45%
Blotchiness 26.32+/−79.75%.
   Formulation containing 3% of Extract 1:
Rash −15.69+/−45.86%
Xerosis −38.64+/−63.33%
Loosening −31.01+/−51.94%
Blotchiness −41.94+/−85.04%.

These results show that the simple application of a cosmetic formulation, whatever it be, is capable of improving the general state of the skin tissue. This improvement can probably be attributed to the hydration obtained after application of the cream. This overall result does not however take into account the <<reactive skin>> parameter studied here and it is suitable to separate the SS and ESS panels for a finer analysis of the results.

When only the sensitive skins are considered, only the formulation containing 3% of an Extract 1 is capable of significantly improving 3 of the 4 clinical signs of skin irritation studied (FIG. 6). As for the placebo formulation, this formulation improves only the <<loosening>> item (FIG. 6), the item most probably in relation with the hydrating effect obtained after the application of a cream.

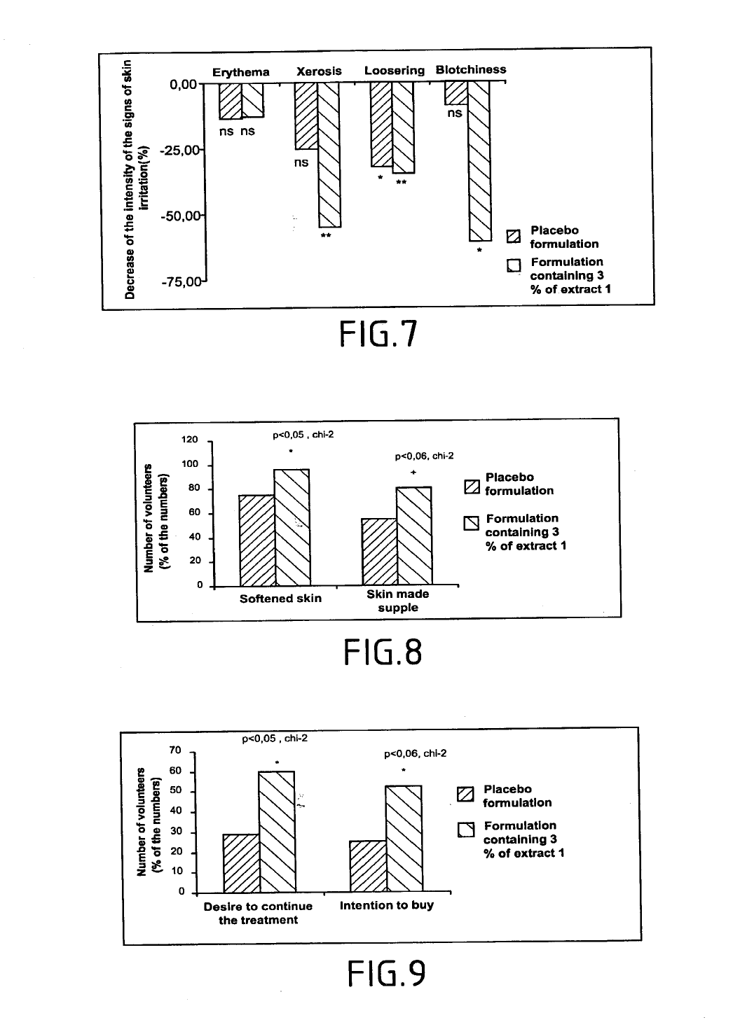

In FIG. 7, an improvement is observed of the signs of skin irritation after 28 days of use of a placebo formulation or a formulation containing 3% of an Extract 1: Analysis of the <<sensitive skin>> sub-groups.

The following comments can be made in relation to FIG. 7
ns: no significant difference between D0 and D28
*: significant difference between D0 and D28 (p<0.05; Wilcoxon's Paired Signed Rank Test)
**: significant difference between D0 and D28 (p<0.01; Wilcoxon's Paired Signed Rank Test)

Percentages of decrease of intensity of the clinical signs of skin irritation:

Placebo Formulation

| | |
|---|---|
| Rash | −13.54 +/− 48.58% |
| Xerosis | −25.48 +/− 97.40% |
| Loosening | −32.11 +/− 54.40% |
| Blotchiness | −9.02 +/− 0.61% |

Formulation containing 3% of INHIPASE®:

| | |
|---|---|
| Rash | −13.00 +/− 47.64% |
| Xerosis | −55.05 +/− 74.91% |
| Loosening | −34.68 +/− 54.78% |
| Blotchiness | −60.61 +/− 117.23% |

In conclusion, Extract 1 is capable of specifically reducing the clinical signs of skin irritation in a population whose skin is particularly reactive. This action being particularly focused on this type of population, Extract 1 seems to constitute a tool of choice in the fight against sensitive skins.

### Questionnaire of Evaluation of the Products.

The questionnaire given to the volunteers having used the placebo formulation or the formulation containing 3% of Extract 1 for 28 days has also enabled demonstrating the significant differences between the products tested (FIGS. 8 and 9).

The formulation containing 3% of Extract 1 enabled a softening of the skin (p<0.05) and a rendering of the skin more supple (p<0.06) significantly greater than that obtained with the placebo formulation (FIG. 8),

In FIG. 8, a softening of the skin and a rendering of the skin more supple are observed in response to the application for 28 days of a placebo formulation or a formulation containing 3% of Extract 1.

The following comments can be made in relation to FIG. 8
*: Significantly different to the placebo group (p<0.05; Chi-2 test)
+: Significantly different to the placebo group (p<0.06; Chi-2 test).

Moreover, the volunteers clearly preferred the formulation containing 3% of Extract 1.(FIG. 9).
→60% of the volunteers desired continuing to use it (versus 29% for the placebo formulation, p<0.05), and
→52% of the volunteers dearly expressed their intent to buy (versus 26% for the placebo formulation, p<0.06).

In FIG. 9, the results of intent to buy and the continuation of the treatment are presented.

The following comments can be made in relation to FIG. 9
*: Significantly different to the placebo group (p<0.05; Chi-2 test)
+: Significantly different to the placebo group (p<0.06; Chi-2 test)
N.B. The Chi-2 test compares the distributions of population. The data are in this graph expressed as number of volunteers, of no use in this case to seek standard deviations.

Extract 1 proposes a specifically-focused action on the reducing of the signs of skin irritation encountered in the subjects having sensitive skins. From this fact, this active constitutes a particularly pertinent tool for relieving reactive skins and for correcting, by cosmetic applications, unpleasant redness and prickling felt by the consumers.

### EXAMPLE 7

### Of the Present Invention

### For of Cosmetic Compositions or of Pharmaceutical Compositions

Use of the products of the invention in formulations of cosmetic compositions or pharmaceutical compositions of oil-in-water emulsion type

### Formulation 7a:

| A | Water | qsp 100 |
|---|---|---|
| | Butylene Glycol | 2 |
| | Glycerin | 3 |
| | Sodium Dihydroxycetyl | 2 |
| | Phosphate, | |
| | Isopropyl Hydroxycetyl Ether | |
| B | Glycol Stearate SE | 14 |
| | Triisononanoin | 5 |
| | Octyl Cocoate | 6 |
| C | Butylene Glycol, | 2 |
| | Methylparaben, | |
| | Ethylparaben, Propylparaben, | |
| | pH adjusted to 5.5 | |
| D | Products of the invention | 0.01-30% |

The phases A and B are heated separately at 75° C., and then B is added to A under vigorous agitation; C and then D are then added, during cooling of the cream thus formed.

### Formulation 7b:

| A | Water | qsp 100 |
|---|---|---|
| | Butylene Glycol | 2 |
| | Glycerin | 3 |
| | Polyacrylamide, Isoparaffin, | 2.8 |
| | Laureth-7 | |
| B | Butylene Glycol, | 2 |
| | Methylparaben, | |
| | Ethylparaben, Propylparaben; | |
| | Phenoxyethanol, | 2 |
| | Methylparaben, | |
| | Propylparaben, Butylparaben, | |
| | Ethylparaben | |
| | Butylene Glycol | 0.5 |
| C | Products of the invention | 0.01-30% |

Phase A is heated to 75° C.; B and then C are added under agitation, during cooling of the formula thus made.

### Formulation 7c:

| | | |
|---|---|---|
| A | Carbomer | 0.50 |
| | Propylene Glycol | 3 |
| | Glycerol | 5 |
| | Water | qsp 100 |
| B | Octyl Cocoate | 5 |
| | Bisabolol | 0.30 |
| | Dimethicone | 0.30 |
| C | Sodium Hydroxide | 1.60 |
| D | Phenoxyethanol, | 0.50 |
| | Methylparaben, | |
| | Propylparaben, Butylparaben, | |
| | Ethylparaben | |
| E | Perfume | 0.30 |
| F | Products of the invention | 0.01-30% |

Phases A and B are heated separately to 75° C., and then B is added to A under vigorous agitation; C and then D and then E and then F are then added, during cooling of the cream thus formed.

### EXAMPLE 8

### Of the Present Invention

Use of the <<anti-inflammatory>> products in a water-in-oil type formulation

| | | |
|---|---|---|
| A | PEG 30 | - 3 |
| | dipolyhydroxystearate | |
| | Capric triglycerides | 3 |
| | Cetearyl Octanoate | 4 |
| | Dibutyl Adipate | 3 |
| | Oil of grape seeds | 1.5 |
| | Oil of Jojoba | 1.5 |
| | Phenoxyethanol, | 0.5 |
| | Methylparaben, | |
| | Propylparaben, Butylparaben, | |
| | Ethylparaben | |
| B | Glycerin | 3 |
| | Butylene Glycol | 3 |
| | Magnesium Sulfate | 0.5 |
| | EDTA | 0.05 |
| | Water | qsp 100 |
| C | Cyclomethicone | 1 |
| | Dimethicone | 1 |
| D | Perfume | 0.3 |
| E | Products of the invention | 0.01-30% |

Phases A and B are heated separately to 75° C., and then B is added to A under vigorous agitation; C and then D and then E are then added, during cooling of the cream thus formed.

### EXAMPLE 9

### Of the Present Invention

Use of the “anti-inflammatory” products in a formulation of face cleaning gel type.

| | | |
|---|---|---|
| A | Xanthan gum | 0.8 |
| | Water | qsp 100 |
| B | Butylene Glycol, | 0.5 |
| | Methylparaben, | |
| | Ethylparaben, Propylparaben | |
| | Phenoxyethanol, | 0.5 |
| | Methylparaben, | |
| | Propylparaben, Butylparaben, | |
| | Ethylparaben | |
| C | Citric add | 0.8 |
| D | Sodium Laureth Sulfate | 40.0 |
| E | Product of the invention | 0.01-30% |

Phases A and B are prepared at ambient temperature separately, and then B is added to A under agitation; C and then D and then E are then added, under moderate agitation.

### EXAMPLE 10

### Of the Present Invention

Use of the products of the invention in an anhydrous type formulation

| | | |
|---|---|---|
| A | Mineral wax | 17.0 |
| | Isostearyl Isostearate | 31.5 |
| | Propylene Glycol Dipelargonate | 2.6 |
| | Propylene Glycol Isostearate | 1.7 |
| | beeswax/PEG 8 | 3.0 |
| | Hydrogenated palm kernel oil | 3.4 |
| | Hydrogenated palm glycerides oil | |
| | Lanolin oil | 3.4 |
| | Sesame oil | 1.7 |
| | Cetyl Lactate | 1.7 |
| | Mineral oil, lanolin alcohol | 3.0 |
| B | Castor oil | Qsp 100 |
| | Products of the invention in dry | 0.001-5% |
| | form | |

Phases A and B are heated separately to 80° C., and then B is added to A under agitation.

### EXAMPLE 11

### Of the Present Invention

Use of the products of the invention in a formulation of aqueous gels (face gels, body gels, etc. . . . )

| A | Water | qsp 100 |
|---|---|---|
| | Carboxyvinyl polymer (also called | 0.5 |
| | carbomer) | |
| | Butylene Glycol | 15 |
| | Phenoxyethanol, Methylparaben, | 0.5 |
| | Propylparaben, Butylparaben, | |
| | Ethylparaben | |
| B | Products of the invention | 0.01-30% |

Phase A is prepared by adding all the ingredients and by heating the whole at 80° C. until a homogenous mixture is obtained. B is then added to A under vigorous agitation during cooling of the gel thus formed.

### EXAMPLE 12

### Harmlessness Test

### Evaluation of the Cosmetic Acceptance of a Preparation Containing the Products of the Invention.

The toxicology tests were carried out on a compound retained, namely Extract 1, used pure, by an ocular evaluation in the rabbit, by the study of the absence of abnormal toxicity by single oral administration in the rat and by the study of the sensitizing ability in the guinea pig.

### 1. Evaluation of the Primary Skin Irritation in the Rabbit:

The preparation described above is applied without dilution at the dose of 0.5 ml on the skin of three rabbits, according to the method recommended by the OECD directive relating to the study of “the acute irritant/corrosive effect on the skin”.

The products are classed according to criteria defined by the Order of Jan. 2, 1982 published in the Journal of the French Republic of 21 Jan. 1982. The results of these tests have enabled concluding that the preparation retained was classed non-irritant for the skin.

### 2. Evaluation of the Ocular Irritation in the Rabbit

The preparation described above was instilled pure, in one batch, at the rate of 0.1 ml, in the eye of three rabbits according to the method recommended by directive of the OECD No. 405 of 24 Feb. 1987 in relation to the study of “the acute irritant/corrosive effect on the eyes”.

The results of this test enable concluding that the preparation can be considered as non-irritant for the eyes, in the sense of Directive 91/326 EEC, used pure.

### 3. Test on the Absence of Abnormal Toxicity by Single Oral Administration in the Rat:

The preparation described was administered in one batch orally at the dose of 5 g/Kg of body weight, to 5 male rats and 5 female rats, according to a protocol inspired by the Directive of the OECD No. 401 of 24 Feb. 1987 and adapted to cosmetic products.

The LD0 and LD50 are found to be greater than 5,000 mg/Kg. The preparation tested is therefore not classed amongst the products which are dangerous by ingestion.

### 4. Evaluation of the Skin Sensitization Potential in the Guinea Pig:

The preparation described is submitted to the maximization test described by Magnusson and Kligmann, a protocol in accordance with the Directive line No. 406 of the OECD.

The preparations are classed as non-sensitizing by skin contact.

### 5. Evaluation of the Phototoxic and Photoallergic Potential by Topical Applications in the Guinea Pig:

10 animals received the product as such in a single topical application, and were then exposed to UV rays so as to appreciate its phototoxic potential.

Then, they received the product to be tested as such by repeated topical applications followed by exposures to UV rays (induction exposures). After a rest period of 10 days, the animals received a single application of the product to be tested as such on unexposed skin followed by an exposure to UV (starting exposure).

In parallel, 5 animals having received the product to be tested under the same conditions and not exposed to UV rays served as irradiation controls.

The evaluation of the phototoxic and photoallergic potential is made by comparison of the intensity of the rashes and of the edemas on the areas treated with the product to be tested and then exposed to UV, and the areas non-treated and exposed to UV, and on the areas treated and non-exposed of the control animals.

Under the experimental conditions adopted, the product tested as such is considered as devoid of phototoxic and photoallergic potential.

We claim:

1. A method of cosmetic care comprising topically applying on the skin of a person in need thereof a cosmetic composition comprising an effective amount of at least one plant extract selected from the group consisting of grape seed extract, Pueraria lobata extract, lemon extract, guarana extract, sunflower extract, liana extract, wherein the plant extract inhibits phospholipase A2 type I or phospholipase A2 type II enzymatic activity.

2. The method of claim 1, wherein the effective amount of the at least one plant extract is between 0.01% and 30% by weight of the total composition.

3. The method of claim 1, wherein the at least one plant extract is a plant extract of Pueraria Lobata root.

4. The method of claim 3, wherein the plant extract of Pueraria Lobata root is in an amount between 1% and 10% by weight of the total composition.

5. The method of claim 3, wherein the plant extract of Pueraria Lobata root is obtained by extracting Pueraria lobata root in an aqueous solvent at a concentration of between 0.1% and 20% by weight.

6. The method of claim 5, wherein the aqueous solvent comprises an alcohol/glycol at a concentration of between 0% and 80% (w/w).

7. The method of claim 6, wherein the alcohol/glycol is butylene glycol.

8. The method of claim 5, wherein the aqueous solvent comprises methyl paraben at a concentration of between 0.01% and 0.5% (w/w).

9. The method of claim 1, wherein the at least one plant extract is a plant extract of grape seeds and comprises butylene glycol and methyl paraben.

10. The method of claim 1, for caring at least one condition selected from the group consisting of irritation, prickling, itching, superficial observation of blotchiness, appearance of small burst vessels, loosening of skin tissues, loss of tone of skin tissues, and dryness of skin tissues.

11. A method of therapeutic treatment comprising topically applying on the skin of a person in need thereof a therapeutic composition comprising an effective amount of at least one plant extract selected from the group consisting of grape seed extract, Pueraria lobata extract, lemon extract, guarana extract, sunflower extract, liana extract, wherein the plant extract inhibits phospholipase A2 type I or phospholipase A2 type II enzymatic activity.

12. The method of claim 11, wherein the effective amount of the at least one plant extract is between 0.01% and 30% by weight of the total composition.

13. The method of claim 11, wherein the at least one plant extract is a plant extract of Pueraria Lobata root.

14. The method of claim 13, wherein the plant extract of Pueraria Lobata root is in an amount between 1% and 10% by weight of the total composition.

15. The method of claim 13, wherein the plant extract of Pueraria Lobata root is obtained by extracting Pueraria lobata root in an aqueous solvent at a concentration of between 0.1% and 20% by weight.

16. The method of claim 15, wherein the aqueous solvent comprises an alcohol/glycol at a concentration of between 0% and 80% (w/w).

17. The method of claim 16, wherein the alcohol/glycol is butylene glycol.

18. The method of claim 15, wherein the aqueous solvent comprises methyl paraben at a concentration of between 0.01% and 0.5% (w/w).

19. The method of claim 11, wherein the at least one plant extract is a plant extract of grape seeds and comprises butylene glycol and methyl paraben.

20. The method of claim 11, for caring at least one condition selected from the group consisting of inflammation, pain, burn, rash, localized blotch of integument linked to an external physical agent, redness of integument linked to an inflammatory syndrome, and xerosis.
